(19) United States
(12) Patent Application Publication
(10) Pub. No.: US 2023/0096140 A1
(43) Pub. Date: Mar. 30, 2023

(54) COAXIAL BIOPSY NEEDLE WITH INTEGRATED STYLET AND ANESTHESIA NEEDLE ARRANGED WITH LOCKING MEANS WITH PREDETERMINED AND DIFFERENT RELEASE TORQUES

(71) Applicant: APRIOMED AB, Uppsala (SE)

(72) Inventor: Beniamino CORBO, Chester Cheshire (GB)

(21) Appl. No.: 17/908,989

(22) PCT Filed: Mar. 4, 2021
(86) PCT No.: PCT/SE2021/050191

§ 371 Date: Sep. 2, 2022

(30) Foreign Application Priority Data
Mar. 4, 2020 (SE) 2050247-2

(51) Int. Cl. A61B 10/02

(52) U.S. Cl. CPC A61B 10/0233; A61B 17/3496

(57) ABSTRACT

The invention relates to coaxial needle assembly (41) for introduction into human tissue, comprising an anesthesia needle (11), a stylet (21) and a coaxial cannula (31), wherein the anesthesia needle is inserted in the stylet and has a distal end (12) that projects out from a distal end (22) of the stylet, and wherein the stylet is inserted in the coaxial cannula and has a distal end (22) that projects out from a distal end (32) of the coaxial cannula, such that, during introduction into and through human tissue, the coaxial needle assembly acts and is manipulated as an integrated device. The assembly is arranged with a predetermined needle-stylet release torque which is required to release the anesthesia needle (11) from the stylet (21), and a predetermined stylet-cannula release torque which is required to release the stylet (21) from the coaxial cannula (31), wherein the value of predetermined needle-stylet release torque is less than the value of predetermined stylet-cannula release torque.

### TECHNICAL FIELD

The present invention relates generally to a medical biopsy device for use in a medical biopsy procedure and relates in particular to a coaxial needle assembly, which comprises a hollow anesthesia needle, a hollow stylet and a hollow coaxial cannula, wherein anesthesia needle is inserted in the hollow stylet, which, in turn, is inserted in the hollow coaxial cannula.

### BACKGROUND

During a medical biopsy procedure an anesthesia needle is first slowly introduced through the tissue overlaying the organ or tissue of interest and is then advanced into the organ or tissue from which a biopsy sample is to be taken. During the penetration an anesthetic is administrated via the hollow anesthesia needle to the tissue being penetrated, to thereby provide anesthesia. This procedure is typically visible on a screen by means of ultrasound or CT imaging. The anesthesia needle is then retracted and removed, and a dilator, e.g. a stylet, is then introduced through the tissue tract created by the anesthesia needle. The doctor who is performing the operation is also for this part typically guided by a visual aid such as ultrasound or CT. The stylet is then removed, and a so-called coaxial cannula is introduced into the no widened tissue tract, to provide a free access to the tissue or organ of interest. Thereafter, a biopsy needle is introduced into the interior of the hollow coaxial cannula, to take a biopsy sample from the tissue or organ of interest.

Although the doctor performing the operation outlined above is guided by a visual aid such as an ultrasound or CT image, there is always a certain risk that stylet during the introduction thereof diverges from the tract created by the anesthesia needle, or that the coaxial cannula diverges from the canal created by the anesthesia needle and the stylet. Clearly, this is a most unwanted event, and an object of the present invention is to provide an improved biopsy device which remedies this risk.

### SUMMARY OF THE INVENTION

The above-mentioned object is achieved with a coaxial biopsy needle assembly according to the independent claim. Preferred embodiments are set forth in the dependent claims. The present invention relates to a coaxial needle assembly for introduction into human tissue, which coaxial needle assembly comprises an anesthesia needle, a stylet and a coaxial cannula, wherein the anesthesia needle is inserted in the stylet and has a distal tip that projects out from a distal end of the stylet, and wherein the stylet is inserted in the coaxial cannula and has a distal tip that projects out from a distal end of the coaxial cannula, such that, during introduction into and through human tissue, the coaxial needle assembly acts and is manipulated as an integrated device.

Since the anesthesia needle is located inside the stylet and has a distal tip the extends out from the distal end of the stylet and the stylet is located in the coaxial cannula and has a distal end the extends out from the distal end of the coaxial needle, the coaxial needle assembly is capable of performing the administration of anesthetic, which is done via the hollow interior of the anesthesia needle, the dilation of the tissue tract and the placement of the coaxial cannula in one step during the insertion of the coaxial needle assembly into the human tissue. In other words, the three steps that previously were performed consecutively with three different devices, i.e. with the anesthesia needle, the stylet and the coaxial cannula, respectively, can now be performed in one step as single procedure. This feature is meant to be encompassed by the expression that “the coaxial needle assembly acts and is manipulated as an integrated device”. When the distal end of the coaxial cannula has been placed at the desired position, the anesthesia needle and the stylet are retracted out from the coaxial cannula and are removed, such that access to the organ or tissue of interest has been created via the hollow interior of the coaxial cannula.

According to one aspect of the invention the coaxial needle assembly comprises an anesthesia needle, a stylet and a coaxial cannula, wherein the anesthesia needle is inserted in the stylet and has a distal end that projects out from a distal end of the stylet, and wherein the stylet is inserted in the coaxial cannula and has a distal end that projects out from a distal end of the coaxial cannula, such that, during introduction into and through human tissue, the coaxial needle assembly acts and is manipulated as an integrated device, wherein the stylet is fixedly but detachably connected to the coaxial cannula and the anesthesia needle is fixedly but detachably connected to the stylet. The coaxial needle assembly is arranged to provide a predetermined needle-stylet release torque which is required to release the anesthesia needle from the stylet, and a predetermined stylet-cannula release torque which is required to release the stylet from the coaxial cannula, and wherein the value of predetermined needle-stylet release torque is less than the value of predetermined stylet-cannula release torque.

According to one embodiment the anesthesia needle at its proximal end is provided with a needle handle and at the distal end of the needle handle is a needle-stylet connector provided and the stylet at its proximal end is provided with a stylet handle and at the distal end of the stylet handle is a stylet-cannula connector provided, and at the proximal end of the stylet handle is a stylet-needle connector provided; and the coaxial cannula at its proximal end is provided with a cannula handle and at the proximal end of the cannula handle is a cannula-stylet connector provided, and wherein the needle-stylet connector of the anesthesia needle is engaged with the stylet-needle connector of the stylet, forming a needle-stylet connector pair, and the stylet-cannula connector of the stylet is engaged with the cannula-stylet connector forming a stylet-cannula connector pair, and wherein the predetermined needle-stylet release torque is provided by the needle-stylet connector being engaged with the stylet-needle connector and the predetermined stylet-cannula release torque is provided by the stylet-cannula connector being engaged with the cannula-stylet connector.

According to one embodiment the stylet-cannula connector pair is arranged to a have a predetermined friction between the cannula-stylet connector and the stylet-cannula connector.

According to one embodiment the needle-stylet connector pair is arranged to a have a predetermined friction between the needle-stylet connector and the stylet-needle connector, wherein friction gives the needle-stylet release torque.

According to one embodiment at least one of the stylet-cannula connector pair and the needle-stylet connector pair comprises a locking arrangement, the locking arrangement comprising a first locking part fixed to one of the connectors in the connector pair and a second locking part fixed to the other connector in the pair, the first locking part which in a first position abutting the second locking part fixed, and wherein in a relative rotational movement between the connectors the first locking part is arranged to pass the second locking part with a predetermined release torque.

According to one embodiment the stylet-cannula connector pair comprises a female connector and a male connector, the female connector and the male connector being arranged with a play in between them, and the female connector is provided with at least one first protrusion and the male connector with at least one second protrusion, and wherein the first protrusion and the second protrusion extends in their respective radial direction so that they overlap in the radial direction, thereby forming a locking arrangement as a rotational lock that is passed by the predetermined stylet-cannula release torque.

According to one embodiment the needle-stylet connector pair comprises a female connector and a male connector, the female connector and the male connector being arranged with a play in between them, and the female connector is provided with at least one first protrusion and the male connector with at least one second protrusion, and wherein the first protrusion and second protrusion extends in their respective radial direction so that they overlap in the radial direction, thereby forming the locking arrangement as a rotational lock that is passed by the predetermined needle-stylet release torque.

According to one embodiment the stylet-cannula connector pair is a bayonet-type coupling comprising a cylindrical male side with a pin and a female receptor with a matching L-shaped slot, the L-shaped slot is provided with a local narrowing which is dimensioned to provide a predetermined force for the pin to pass, giving the stylet-cannula release torque.

According to one embodiment the stylet-cannula needle-stylet connector pair is a bayonet-type coupling comprising a cylindrical male side with a pin and a female receptor with a matching L-shaped slot, the L-shaped slot is provided with a local narrowing which is dimensioned to provide a predetermined force for the pin to pass, giving the needle-stylet release torque.

According to one embodiment the stylet-cannula connector pair is provided with a breakable fixture provided in, the breakable fixture being arranged to break at the predetermined stylet-cannula release torque and thereby forming a one-time locking arrangement is provided.

According to one embodiment the value of the needle-stylet release torque is at least 20% lower than the value of the stylet-cannula release torque and even more preferably the value of the needle-stylet release torque is at least 50% lower than the value of the stylet-cannula release torque. The stylet-cannula release torque is preferably arranged to be between 0.08 and 0.15 Nm and even more preferably between 0.1 and 0.12 Nm.

According to one embodiment the anesthesia needle is slidable within the stylet.

According to one embodiment the distal end of the anesthesia needle projects 10-30 mm out from the distal end of the stylet.

According to one embodiment the distal end portion of the anesthesia needle is provided with at least one ecogenic marking.

According to one embodiment the distal tip of the stylet is provided with a so-called trocar grind, which is a way of grinding the stylet tip such that three facets are formed, which provides for good penetration ability of the coaxial needle assembly.

Thanks to the invention the operator may control the coaxial needle assembly more precise and secure compared with prior art devices.

One advantage of the present inventions is that the needle-stylet release torque and the stylet-cannula release torque, and in particular their relation may be provided with a high degree of control and repeatability. Thereby the operator will always have the same experience in using different samples of the coaxial needle assembly according to the invention. By providing different torques the needle-stylet release torque and the stylet-cannula release torque, the risk of releasing the stylet from the cannula while operating the needle is reduced. In addition the operator is provided with a tactile difference between the connector pairs.

One advantage of using a locking arrangement in the connectors is that with such arrangement the predetermined torques will be independent of factors such as humidity and temperature.

### BRIEF DESCRIPTION OF THE DRAWINGS

The present invention will be further explained hereinafter by means of non-limiting examples and with reference to the appended drawings, wherein:

FIG. 1 illustrates a hollow anesthesia needle, which is part of a coaxial needle assembly according to the present invention;

FIG. 2 illustrates a hollow stylet, which is part of the coaxial needle assembly according to the present invention;

FIG. 3 illustrates a hollow coaxial cannula, which is part of the coaxial needle assembly according to the present invention;

FIG. 4 illustrates the coaxial needle assembly according to the present invention, which coaxial needle assembly comprises the anesthesia needle of FIG. 1, the stylet of FIG. 2 and the coaxial cannula of FIG. 3;

FIG. 5a-b illustrate embodiments of connectors according to the present invention; and

FIG. 6 illustrates an embodiment of a connector according to the present invention.

### DETAILED DESCRIPTION

In the figures, same, similar or corresponding elements have been given the same reference numerals.

FIG. 1 illustrates an anesthesia needle 11, which is hollow and has a distal end 12 and a proximal end 13 and which is part of a coaxial needle assembly 41 illustrated in FIG. 4. To be clearly visible on an ultrasound image, a distal end portion of the anesthesia needle 11 (i.e. a portion that extends a short distance proximally of the distal end 12 of the anesthesia needle 11) can be provided with one or several ecogenic markings, which are not visible in FIG. 1. The anesthesia needle 11 is at its proximal end provided with a needle handle 131. At the distal end of the needle handle 131 is a needle-stylet connector 133 provided. Between the proximal end 13 of the anesthesia needle 11 and the needle-stylet connector 133 is a needle grip portion 132 provided. The needle grip portion 132 is during use intended to be gripped by the operator's fingertips, typically between the thumb and the index finger of the operator. The needle grip portion 132 may therefore be textured or provided with groves, ribs, or notches to improve the grip. A further alternative, which is depicted, is to provide the needle handle 131 with one or more protrusions, for example a pair of wing-like protrusions 134 extending radially out from an otherwise cylindrical needle handle 131.

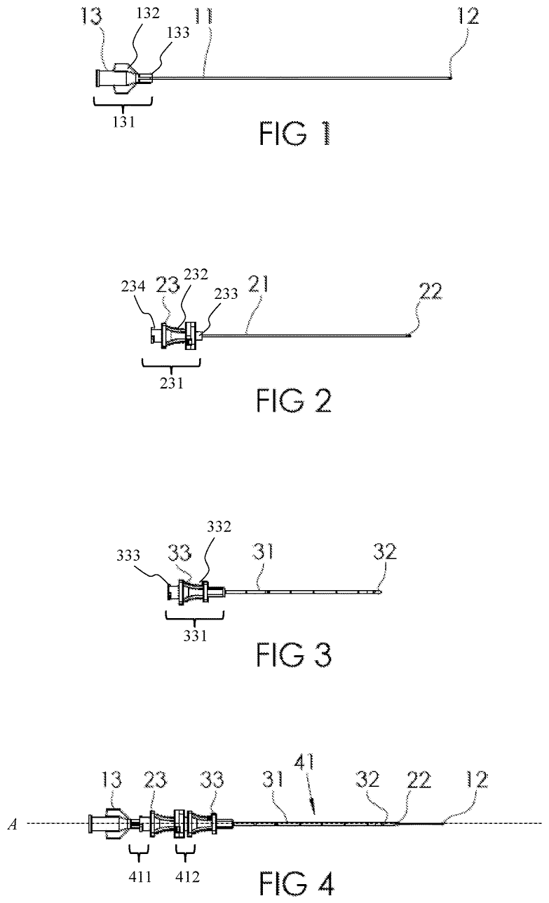

FIG. 2 illustrates a stylet 21, which is hollow and has a distal end 22 and a proximal end 23 and which is part of the coaxial needle assembly 41 illustrated in FIG. 4. In a preferred embodiment, the distal end 22 is provided with a so-called trocar grind, which provides three facets at the distal end 22 and which ensures a good penetration ability for the stylet 21 and therefore for the coaxial needle assembly 41. The stylet 21 is at its proximal end 23 provided with a stylet handle 231. At the distal end of the stylet handle 231 is a stylet-cannula connector 233 provided. At the proximal end of the stylet handle 231 is a stylet-needle connector 234 provided. Between the stylet-cannula connector 233 and the stylet-needle connector 234 is a stylet grip portion 232 provided. The stylet grip portion 232 is during use intended to be gripped by the operator's fingertips, typically between the thumb and the index finger of the operator. The stylet grip portion 232 may therefore be textured or provided with groves, ribs, or notches to improve the grip. A further alternative is to provide the stylet handle 231 with one or more protrusions, for example a pair of wing-like protrusions extending radially out from the stylet handle 231.

FIG. 3 illustrates a coaxial cannula 31, which is hollow and has a distal end 32 and a proximal end 33 and which is part of the coaxial needle assembly 41 illustrated in FIG. 4. The coaxial cannula 31 is at its proximal end provided with a cannula handle 331. At the proximal end of the cannula handle 331 is a cannula-stylet connector 333 provided. Between the cannula-stylet connector 333 and the coaxial cannula 31 is a cannula grip portion 332 provided. The cannula grip portion 332 is during use intended to be gripped by the operator's fingertips, typically between the thumb and the index finger of the operator. The cannula grip portion 332 may therefore be textured or provided with groves, ribs, or notches to improve the grip.

The anesthesia needle 11, the stylet 21 and the coaxial cannula 31 have all basically conventional designs, but their specific dimensions and connection couplings are adapted to function and co-operate together as an integrated coaxial needle assembly 41 according to the invention, as will be explained below.

In FIG. 4, the coaxial needle assembly 41 is shown, and it can in particular be seen that the anesthesia needle 11 has been inserted into the stylet 21 such that the distal end 12 of the anesthesia needle 11 projects a distance out of the distal end 22 of the stylet 21. In preferred embodiments of the invention, the distance that the distal end 12 of the anesthesia needle 11 protrudes from the distal end 22 of the stylet 21 can be in the interval of about 10-30 mm, which is a suitable distance for ensuring that the tissue which the distal end 12 penetrates is adequately anesthetized while the coaxial needle assembly 41 being slowly advanced through the tissue. It can further be seen that the stylet 21 has been inserted into the coaxial cannula 31 such that the distal end 22 of the stylet 21 extends a short distance out from the distal end 32 of the coaxial cannula 31, to ensure that the tissue being penetrated is gradually widening to accommodate the full circumferential size of the coaxial cannula 31. The longitudinal relationships between the anesthesia needle 11, the stylet 21 and the cannula 31 mounted into the coaxial needle assembly 41 are given by the interactions between the needle handle 131 and the stylet handle 231 and between the stylet handle 231 and the cannula handle 331, respectively. In the coaxial needle assembly 41 the needle-stylet connector 133 of the anesthesia needle 11 is engaged with the stylet-needle connector 234 of the stylet 21, forming a needle-stylet connector pair 411. The stylet-cannula connector 233 of the stylet 21 is engaged with the cannula-stylet connector 333 forming a stylet-cannula connector pair 412. The coaxial needle assembly 41 has a common rotational axis A for the stylet-cannula connector pair 412 and the needle-stylet connector pair 411.

A needle-stylet release torque can be ascribed to the needle-stylet connector pair 411 defining the torque required to release the needle handle 131 from the stylet handle 231 and thereby the anesthesia needle 11 from the stylet 21, by turning the needle grip portion 132 in relation to the stylet grip portion 232. The turning motion in normal use associated to the operator performing a screwing motion with his thumb and index finger, while holding the cannula grip portion 332 still, for example using the remaining fingers and the palm. A needle-stylet release force will be associated with the release torque and the radial distance from the center axis A to the point on the needle grip portion 132 there the force is applied. If, for example, the needle grip portion 132 is essentially cylindrical, the radial distance will effectively be the radius of the cylindrical needle grip portion 132. If the needle grip portion 132 is provided with protrusions, for example the wing-like structure depicted in FIG. 1, the radial distance will be given by the intended point of contact with the finger or fingers with the protrusion.

Similar to above, a stylet-cannula release torque can be ascribed to the stylet-cannula connector pair 412 defining the torque required to release the stylet handle 231 from the cannula handle 331 and thereby the stylet 21 from the coaxial cannula 31, by turning the stylet grip portion 232 in relation to the cannula grip portion 332. A stylet-cannula release force will be associated with the release torque and the radial distance from the center axis A to the point on the stylet grip portion 232 there the force is applied.

According to one aspect of the invention the needle-stylet release torque of the needle-stylet connector pair 411 is smaller than the stylet-cannula release torque of the stylet-cannula connector pair 412. Preferably, the value of the needle-stylet release torque is at least 20% lower than the value of the stylet-cannula release torque and even more preferably the value of the needle-stylet release torque is at least 50% lower than the value of the stylet-cannula release torque. Preferably the stylet-cannula release torque is between 0.08 and 0.15 Nm and even more preferably between 0.1 and 0.12 Nm. Preferably the needle-stylet release torque is between 0.04 and 0.12 Nm and even more preferably between 0.08 and 0.1 Nm.

The requirement that the stylet-cannula release torque of the stylet-cannula connector pair 412 should be greater than the needle-stylet release torque of the needle-stylet connector pair 411 and preferably with a margin, ensures that during operation the stylet 21 will not unintentionally be released from the cannula 31 while the needle handled 131 is operated, i.e. then the anesthesia needle 11 is released from the stylet 21 by relative turning motion of the needle handle 131 vis-à-vis the stylet handle 231. In addition, by providing different release torques to the stylet-cannula connector pair 412 and the needle-stylet connector pair 411 the operator is afforded a tactile difference in the handling of the needle grip portion 132 and the stylet grip portion 232, respectively.

According to one embodiment the stylet-cannula connector pair 412 is arranged to a have a predetermined friction between the cannula-stylet connector 333 and the stylet-cannula connector 233, wherein the friction gives the stylet-cannula release torque. As appreciated by the skilled person a specific friction between the cannula-stylet connector 333 and the stylet-cannula connector 233 may be provided by selecting materials and the design of the connector parts.

According to one embodiment the needle-stylet connector pair 411 is arranged to a have a predetermined friction between the needle-stylet connector 133 and the stylet-needle connector 234, wherein friction gives the needle-stylet release torque. A specific friction may be provided in the manner described above.

The skilled person may, faced with the requirements of the two different release torques and their relation adjust the tolerances of the molds typically used for fabricating the parts in order to achieve the desired release torques and their relation.

According to one embodiment at least one of the stylet-cannula connector pair 412 and the needle-stylet connector pair 411 is a connector comprising a female part and a male part in a tight fit arrangement and the predetermined friction is provided between the inner surface of the female part and the outer surface of the male part. If one or both connector pairs are of Luer-type or any other type of threaded connector the friction would typically originate from the interacting threads. Similar as with the tight fit embodiment, the skilled person would know how to select materials and/or threading to provide a predetermined friction and hence a predetermined release torque. According to one embodiment both the stylet-cannula connector pair 412 and the needle-stylet connector pair 411 are Luer-type connectors. According to one embodiment at least one of the stylet-cannula connector pair 412 and the needle-stylet connector pair 411 is of Luer-type. According to one embodiment the needle-stylet connector pair 411 is a tight fit connector and the stylet-cannula connector pair 412 is of Luer-type. The desired respective release torques may have been accomplished by carefully adjusting the respective molds to provide the friction giving the torques.

According to one embodiment at least one of the stylet-cannula connector pair 412 and the needle-stylet connector pair 411 comprises a locking arrangement. The locking arrangement comprises a first locking part fixed to one of the connectors in the connector pair which in a first position abuts a second locking part fixed to the other connector in the pair. In a relative rotational movement between the connectors the first locking part passes the second locking part and wherein the interaction between the first locking part and the second locking part defines the release torque. In the following, the figures illustrating various locking arrangements the size of the locking arrangement parts as well as the play between the connector parts are greatly exaggerated. This is to illustrate their function only.

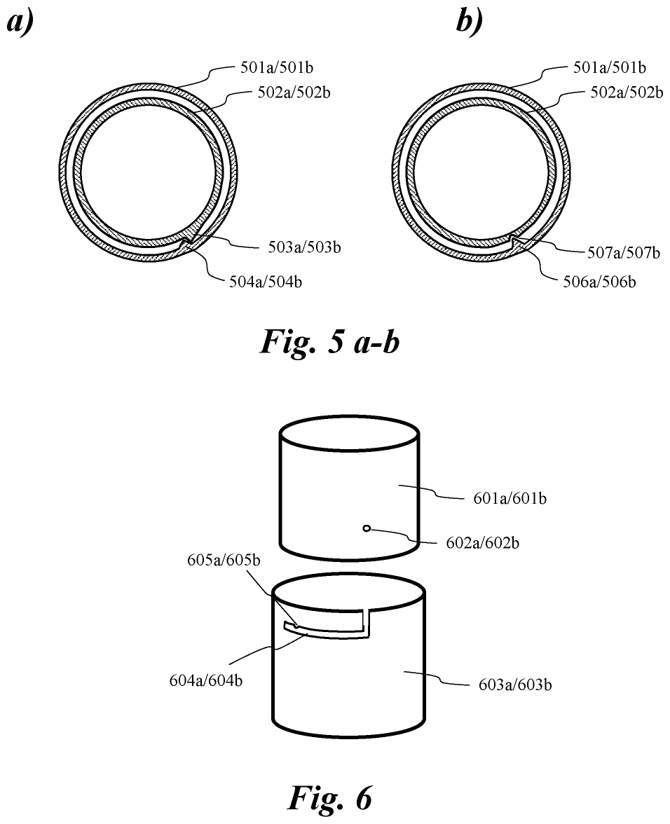

According to one embodiment, schematically illustrated in a cross-sectional view in FIG. 5a-b, the stylet-cannula connector pair 412 comprises a female connector 501a and a male connector 502a. The female connector 501a may be a part of the stylet handle 231 and the male connector 502a a part of the cannula handle 331. Alternatively, the female connector 501a is a part of the cannula handle 331 and the male connector 502a a part of the stylet handle 231. The female connector 501a and the male connector 502a are arranged with a play in between them. The female connector 501a is provided with at least one first protrusion 503a and the male connector 502a with at least one second protrusion 504a. The first protrusion 503a and second protrusion 504a extending in their respective radial direction so that they overlap in the radial direction, thereby forming the locking arrangement as a rotational lock that is passed by a predetermined torque, the stylet-cannula release torque. In an alternative embodiment, schematically illustrated in FIG. 5b the female connector 501a or the male connector 502a is provided with a protrusion 506a and the other connector with a corresponding recess 507a. In the locked position of the stylet-cannula connector pair 412, the protrusion 506a rests in the recess 507a and the stylet-cannula release torque is the torque required to move the protrusion 506a from the recess 507a. The protrusions 503a, 504a, 506a and/or the recess 507a may extend only a short distance in the longitudinal direction of respective handle. Thereby, if the connector is threaded or if the rotational relative moment gives a simultaneously longitudinal movement by other means, the interaction between the protrusions or protrusion-recess may be designed to occur only once during the loosening of the connector. This is for example achieved by having only one protrusion-protrusion/recess pair and letting the longitudinal extension of the protrusions/recesses to be shorter than the longitudinal distance that the female connector 501a and the male connector 502a are separated during one turn.

According to one embodiment, also schematically illustrated in a cross-sectional view in FIG. 5a-b, the needle-stylet connector pair 411 comprises a female connector 501b and a male connector 502b. The female connector 501b may be a part of the needle handle 131 and the male connector 502b a part of stylet handle 231. Alternatively, the female connector 501b is a part of the stylet handle 231 and the male connector 502b a part of the needle handle 131. The female connector 501b and the male connector 502b are arranged with a play in between them. The female connector 501b is provided with at least one first protrusion 503b and the male connector 502b with at least one second protrusion 504b. The first protrusion 503b and second protrusion 504b extending in their respective radial direction so that they overlap in the radial direction, thereby forming the locking arrangement as a rotational lock that is passed by a predetermined torque, the needle-stylet release torque. In an alternative embodiment, schematically illustrated in FIG. 5b the female connector 501b or the male connector 502b is provided with a protrusion 506b and the other connector with a corresponding recess 507b. In the locked position of the stylet-cannula connector pair 412, the protrusion 506b rests in the recess 507b and the needle-stylet release torque is the torque required to move the protrusion 506b from the recess 507b. The protrusions 503b, 504b, 506b and/or the recess 507b may extend only a short distance in the longitudinal direction of respective handle. Thereby, if the connector is threaded or if the rotational relative moment gives a simultaneously longitudinal movement by other means, the interaction between the protrusions or protrusion-recess may be designed to occur only once during the loosening of the connector. This is for example achieved by having only one protrusion-protrusion/recess pair and letting the longitudinal extension of the protrusions/recesses to be shorter than the longitudinal distance that the female connector 501b and the male connector 502b separate during one turn.

One embodiment, schematically illustrated in a perspective side-view in FIG. 6, wherein, for clarity of the illustration, only the connector pair parts are illustrated in the figure. The stylet-cannula connector pair 412 is a bayonet-type coupling comprising a cylindrical male side 601a with a pin 602a and a female receptor 603a with a matching L-shaped slot 604a or grove. The locking arrangement is provided by providing a local narrowing 605a of the slot 604a which requires a predetermined force for the pin 602a to pass, giving the stylet-cannula release torque.

According to one embodiment, also schematically illustrated in a side view in FIG. 6, the needle-stylet connector pair 411 is a bayonet-type coupling comprising a cylindrical male side 601b with a pin 602b and a female receptor 603b with a matching L-shaped slot 604b or grove. The locking arrangement is provided by providing a local narrowing 605b of the slot 604b which requires a predetermined force for the pin 602b to pass, giving the needle-stylet release torque. As realized by the skilled person a bayonet type connector may be realized in many different ways. For example, may the pin or pins be provided on the female receptor and the L-shaped slot on the male side; The slot may not be a sharp L-shape rather have a smooth curvature. Such variations are within the scope of the invention.

According to one embodiment the locking arrangement is formed by a breakable fixture provided in the stylet-cannula connector pair 412, the breakable fixture for example being glue or a local deformation caused by an indentation or by melting. A breakable fixture may also be provided on the outside of the stylet-cannula connector pair 412 by applying a piece of tape or plastic film. The breakable fixture is arranged to break or give in at the predetermined stylet-cannula release torque and thereby a one-time locking arrangement is provided.

According to one embodiment only the stylet-cannula connector pair 412 is provided with a locking arrangement and the needle-stylet connector pair 411 is of tight fit type or Luer-type.

In the coaxial needle assembly according 41 to the present invention, the anesthesia needle 11, the stylet 21 and the coaxial cannula 31 can all have sizes that are commonly used today, and the anesthesia needle 11 can, for example, have a size in the interval of 20-25 G, where G is the Birmingham gauge number, which in medicine specifies the outside diameter of devices like suture wires, cannulae, catheters and needles, where the highest gauge number is 36, which corresponds to the smallest diameter of 0.10 mm. The coaxial cannula 31 can, for example, have a size in the interval of 10-19 G, and the stylet 21 can, for example, have a size in the interval of 11-24 G. In practice, the respective sizes of the anesthesia needle 11, the stylet 21 and the coaxial cannula 31 are selected such that there is a snug fit between the anesthesia needle 11 and the stylet 21 and between the stylet 21 and the coaxial cannula 31.

The proximal end 23 of the stylet 21 is fixedly but detachably connectable to the proximal end 33 of the coaxial cannula 31 by, for example, a bayonet coupling or a luer coupling, where the latter is almost standard within the field of medical devices. The proximal end 13 of the anesthesia needle 11 can be fixedly but detachably connectable to the proximal end 23 of the stylet 21 by, for example a bayonet or luer coupling. It may, however, be preferred that the anesthesia needle 11 only is inserted in the stylet 21 in a tight fit, such that the anesthesia needle 11 can slide backwards within the stylet 21 in case the distal end 12 of the anesthesia needle 11 encounters hard tissue, such as bone, when the coaxial needle assembly 41 is in operation and is penetrating tissue. In this case, the doctor may hold a finger on the proximal end 13 of the anesthesia needle 11 to secure that the anesthesia needle 11 is in place within the stylet 2 during the medical operation.

Although the present invention has been described with reference to specific embodiments, also shown in the appended drawings, it will be apparent to those skilled in the art that many variations and modifications can be done within the scope of the invention as described in the specification and defined with reference to the claims below.

1. A coaxial needle assembly (41) for introduction into human tissue, comprising an anesthesia needle (11), a stylet (21) and a coaxial cannula (31), wherein the anesthesia needle (11) is inserted in the stylet (21) and has a distal end (12) that projects out from a distal end (22) of the stylet (21), and wherein
the stylet (21) is inserted in the coaxial cannula (31) and has a distal end (22) that projects out from a distal end (32) of the coaxial cannula (31), such that, during introduction into and through human tissue, the coaxial needle assembly (41) acts and is manipulated as an integrated device,
the stylet (21) is fixedly but detachably connected to the coaxial cannula (31) and the anesthesia needle (11) is fixedly but detachably connected to the stylet (21),
a predetermined needle-stylet release torque is required to release the anesthesia needle (11) from the stylet (21), and a predetermined stylet-cannula release torque is required to release the stylet (21) from the coaxial cannula (31), and
the value of predetermined needle-stylet release torque is less than the value of predetermined stylet-cannula release torque.

2. The coaxial needle assembly (41) according to claim 1, wherein
the anesthesia needle (11) at its proximal end is provided with a needle handle (131) and at the distal end of the needle handle (131) is a needle-stylet connector (133) provided;
the stylet (21) at its proximal end (23) is provided with a stylet handle (231) and at the distal end of the stylet handle (231) is a stylet-cannula connector (233) provided, and at the proximal end of the stylet handle (231) is a stylet-needle connector (234) provided;
the coaxial cannula (31) at its proximal end is provided with a cannula handle (331) and at the proximal end of the cannula handle (331) is a cannula-stylet connector (333) provided;
the needle-stylet connector (133) of the anesthesia needle (11) is engaged with the stylet-needle connector (234) of the stylet (21), forming a needle-stylet connector pair (411), and the stylet-cannula connector (233) of the stylet (21) is engaged with the cannula-stylet connector (333) forming a stylet-cannula connector pair (412); and
the predetermined needle-stylet release torque is provided by the needle-stylet connector (133) being engaged with the stylet-needle connector (234) and the predetermined stylet-cannula release torque is provided by the stylet-cannula connector (233) being engaged with the cannula-stylet connector (333).

3. The coaxial needle assembly (41) according to claim 2, wherein the stylet-cannula connector pair (412) is arranged to a have a predetermined friction between the cannula-stylet connector (333) and the stylet-cannula connector (233).

4. The coaxial needle assembly (41) according to claim 2, wherein the needle-stylet connector pair (411) is arranged to a have a predetermined friction between the needle-stylet connector (133) and the stylet-needle connector (234), and friction gives the needle-stylet release torque.

5. The coaxial needle assembly (41) according to claim 2, wherein
at least one of the stylet-cannula connector pair (412) and the needle-stylet connector pair (411) comprises a locking arrangement, the locking arrangement comprising a first locking part fixed to one of the connectors in the connector pair and a second locking part fixed to the other connector in the pair, the first locking part which in a first position abutting the second locking part fixed, and
in a relative rotational movement between the connectors the first locking part is arranged to pass the second locking part with a predetermined release torque.

6. The coaxial needle assembly (41) according to claim 2, wherein
the stylet-cannula connector pair (412) comprises a female connector (501a) and a male connector (502a), the female connector (501a) and the male connector (502a) being arranged with a play in between them, and the female connector (501a) is provided with at least one first protrusion (503a) and the male connector (502a) with at least one second protrusion (504a), and
the first protrusion (503a) and the second protrusion (504a) extends in their respective radial direction so that they overlap in the radial direction, thereby forming a locking arrangement as a rotational lock that is passed by the predetermined stylet-cannula release torque.

7. The coaxial needle assembly (41) according to claim 2, wherein
the needle-stylet connector pair (411) comprises a female connector (501b) and a male connector (502b), the female connector (501b) and the male connector (502b) being arranged with a play in between them, and the female connector (501b) is provided with at least one first protrusion (503b) and the male connector (502b) with at least one second protrusion (504b), and
the first protrusion (503b) and second protrusion (504b) extends in their respective radial direction so that they overlap in the radial direction, thereby forming the locking arrangement as a rotational lock that is passed by the predetermined needle-stylet release torque.

8. The coaxial needle assembly (41) according to claim 2, wherein the stylet-cannula connector pair (412) is a bayonet-type coupling comprising a cylindrical male side (601a) with a pin (602a) and a female receptor (603a) with a matching L-shaped slot (604a), the L-shaped slot is provided with a local narrowing (605a) which is dimensioned to provide a predetermined force for the pin (602a) to pass, giving the stylet-cannula release torque.

9. The coaxial needle assembly (41) according to claim 2, wherein the stylet-cannula needle-stylet connector pair (411) is a bayonet-type coupling comprising a cylindrical male side (601a) with a pin (602a) and a female receptor (603a) with a matching L-shaped slot (604a), the L-shaped slot is provided with a local narrowing (605a) which is dimensioned to provide a predetermined force for the pin (602a) to pass, giving the needle-stylet release torque.

10. The coaxial needle assembly (41) according to claim 2, wherein the stylet-cannula connector pair 412 is provided with a breakable fixture provided in, the breakable fixture being arranged to break at the predetermined stylet-cannula release torque and thereby forming a one-time locking arrangement is provided.

11. The coaxial needle assembly (41) according to claim 1, wherein the value of the needle-stylet release torque is at least 20% lower than the value of the stylet-cannula release torque and even more preferably the value of the needle-stylet release torque is at least 50% lower than the value of the stylet-cannula release torque.

12. The coaxial needle assembly (41) according to claim 11, wherein the stylet-cannula release torque is arranged to be between 0.08 and 0.15 Nm and even more preferably between 0.1 and 0.12 Nm.

13. The coaxial needle assembly (41) according to claim 1, wherein the anesthesia needle (11) is slidable within the stylet (21).

14. The coaxial needle assembly (41) according to claim 1, wherein the distal end (12) of the anesthesia needle (11) projects 10-30 mm out from the distal end (22) of the stylet (21).

15. The coaxial needle assembly (41) according to claim 1, wherein a distal end portion of the anesthesia needle (11) is provided with at least one ecogenic marking.

16. The coaxial needle assembly (41) according to claim 3, wherein the needle-stylet connector pair (411) is arranged to a have a predetermined friction between the needle-stylet connector (133) and the stylet-needle connector (234), and friction gives the needle-stylet release torque.

17. The coaxial needle assembly (41) according to claim 2, wherein the value of the needle-stylet release torque is at least 20% lower than the value of the stylet-cannula release torque and even more preferably the value of the needle-stylet release torque is at least 50% lower than the value of the stylet-cannula release torque.

18. The coaxial needle assembly (41) according to claim 3, wherein the value of the needle-stylet release torque is at least 20% lower than the value of the stylet-cannula release torque and even more preferably the value of the needle-stylet release torque is at least 50% lower than the value of the stylet-cannula release torque.

19. The coaxial needle assembly (41) according to claim 4, wherein the value of the needle-stylet release torque is at least 20% lower than the value of the stylet-cannula release torque and even more preferably the value of the needle-stylet release torque is at least 50% lower than the value of the stylet-cannula release torque.

20. The coaxial needle assembly (41) according to claim 5, wherein the value of the needle-stylet release torque is at least 20% lower than the value of the stylet-cannula release torque and even more preferably the value of the needle-stylet release torque is at least 50% lower than the value of the stylet-cannula release torque.
